(19) United States
(12) Plant Patent Application Publication
(10) Pub. No.: US 2003/0046745 P1
(43) Pub. Date: Mar. 6, 2003

(54) GUZMANIA PLANT NAMED 'DEPLAKAR'

(76) Inventor: Hubertus L. van Schie, Delfgauw (NL)

Correspondence Address: Richard C. Peet, Foley & Lardner, Washington Harbour, Suite 500, 3000 K Street, N.W., Washington, DC, 20007-5143, US

(21) Appl. No.: 10/095,443

(22) Filed: Mar. 13, 2002

(30) Foreign Application Priority Data
Aug. 28, 2001 (EP) 2001/1359

(51) Int. Cl. A01H 5/00

(52) U.S. Cl. PLT/371

(57) ABSTRACT

A new and distinct Guzmania plant named ‘Deplakar’ characterized by compact plant shape; short leaves; compact flower shape; yellow inflorescences with red/orange tips.

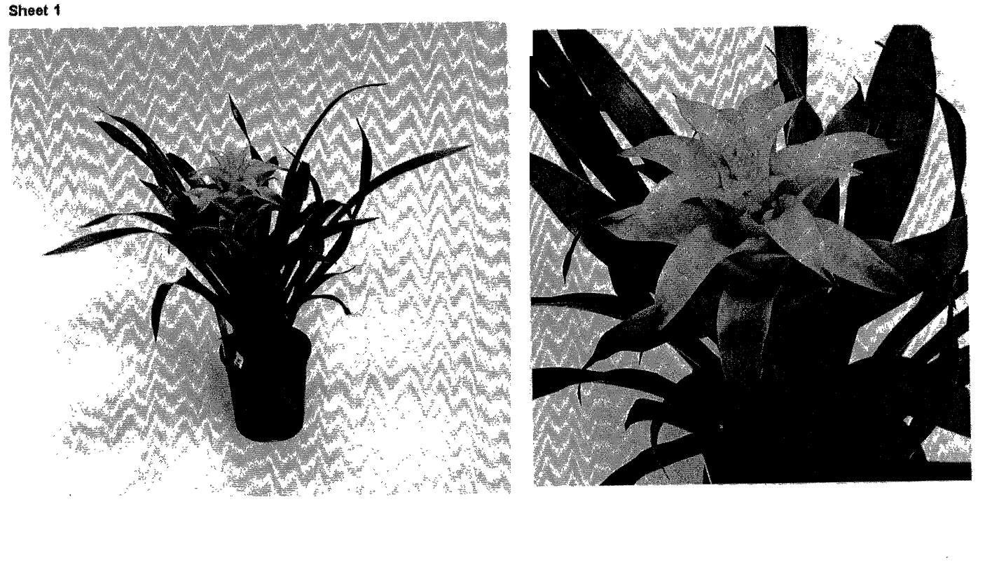

### BACKGROUND OF THE INVENTION

The present invention comprises a new and distinct cultivar of Guzmania plant, hereinafter referred to by the cultivar name ‘Deplakar’. The genus Guzmania is a member of the family Bromeliaceae.

Guzmania comprise a genus of over 100 species of herbaceous evergreen perennials suitable for cultivation in the home or under glass. Guzmania are predominantly epiphytic with a few terrestrial species and are native to the tropics. For the most part the species vary in diameter from 7 or 8 inches to 3 or 4 feet and have rosettes of glossy, smooth edged leaves.

Floral bracts of Guzmania frequently have brilliant colors and may last for many months. The range of flower colors for Guzmania is generally from the yellow through orange but may also include flame red and red-purple. White or yellow, tubular, three petalled flowers may also appear on a stem or within the leaf rosette but are usually short lived.

Guzmania may be advantageously grown as potted plants for greenhouse or home use. Desirably the plants are shaded from direct sunlight during the spring to autumn period, the central vase-like part of the leaf rosette is normally filled with water.

Guzmania is native to tropical America. Leaves of the Guzmania are usually formed as basal rosettes which are stiff and entire and in several vertical ranks. Guzmania have terminal spikes or panicles which are often bracted with petals united in a tube about as long as the calyx.

Asexual propagation of Guzinania is frequently done through the use of tissue culture practices. Propagation can also be from off-shoots which are detached from the mother plant, and may be grown in an appropriate soil or bark mixture.

The new cultivar ‘Deplakar’ is the product of a mutant and was originated by the inventor Hubertus L. van Schie, in 1998 in Delfgauw, The Netherlands. The parent is Guzmania ‘Marjan’ (unpatented). The selection comprising the new variety was chosen after commencement of flowering in 1998.

The new cultivar was asexually propagated by taking cuttings by Deroose Plants in Evergem, Belgium in 1998. Asexual propagation by tissue culture was initiated in 1999. Continuous asexual propagation has demonstrated that the combination of characteristics as herein disclosed for the new cultivar ‘Deplakar’are firmly fixed and are retained through successive generations of asexual reproduction. The new cultivar reproduces true to type.

### BRIEF SUMMARY OF THE INVENTION

The following traits have been repeatedly observed and are determined to be basic characteristics of ‘Deplakar’ which in combination distinguish this Guzmania as a new and distinct cultivar:

1. compact plant shape;

2. short leaves;

3. compact flower shape;

4. yellow inflorescences with red/orange tips; and

5. green leaves.

‘Deplakar’ has not been observed under all possible environmental conditions. The phenotype of the new cultivar may vary significantly with variations in environment such as temperature, light intensity, and daylength without any change in genotype.

Of the many commercial cultivars known to the present inventor, the most similar in comparison to ‘Deplakar’ is the parental cultivar ‘Marjan’ which is characterized by its yellow inflorescence with red/orange tips. In comparison to ‘Marjan’, the inflorescences, leaves, and plant shape of ‘Deplakar’ are more compact.

### BRIEF DESCRIPTION OF THE DRAWING

The accompanying photographic illustrations shows a 19-month-old ‘Deplakar’ plant propagated by tissue culture following growth under appropriate growing conditions, with colors being as true as possible with illustrations of this type. The first drawing shows a ‘Deplakar’ plant on the left and a close-up view of the inflorescence and foliage characteristics to the right. The second drawing illustrates the differences between ‘Deplakar’ (right) and ‘Marjan’ (left).

### DETAILED BOTANICAL DESCRIPTION

The following observations, measurements and values describe plants grown in Delfgauw, The Netherlands under greenhouse conditions which closely approximate those generally used in horticultural practice. ‘Deplakar’ is grown in a commercial greenhouse under 21 degrees Celsius day and night. No artificial lighting or photoperiodic treatments are conducted but ‘Deplakar’ is forced into flowering by adding acethylene. Highest temperature resistance is 40 degrees Celsius, the lowest 5 degrees Celsius. Direct sunlight has to be avoided because it causes burning of the leaves. The following fertilizer is added: 1 part nitrogen, 0.5 parts phosphor. 3 parts Kalium and 0.2 parts Magnesium. Water should not contain too much salts. From the start of tissue culture it takes five years to produce a commercial plant. The amount of time needed to produce a flower depends on the amount of acethylene is added. The flowers have a tenability of up to six months depending on the environment they are grown and kept in.

Color references are made to The Royal Horticultural Society Colour Chart (RHS), except where general colors of ordinary significance are used.

Classification:

Genus.—Guzmania.

Commercial.—Guzmania c.v. Deplakar.

Parentage:

Mutant parent.—Guzmania ‘Marjan’.

Propagation: By tissue culture

Plant:

Form.—Upright, leaves in basal rosette.

Height.—Average 35 cm.

Diameter.—Average 60 cm.

Growth habit.—Upright, growth moderate.

FOliage:

Size.—Leaves have an average length of 39 cm and an average width of 3.5 cm (measured at the middle of a leaf). Leaves are variable in width (even on one plant); width of mature leaf varies from 2.3 cm to 4.3 cm.

Shape.—Linear to narrow linear, tips acuminate to long acute.

Surface texture.—Smooth, glossy.

Leaf color.—Upper side yellow-green, closest to but greener than RHS 146A; under side yellow-green, RHS 146A.

Bracts:

Length/Width/Color.—Length average 9.5 cm; width average 3.4 cm (measured at ⅓ from the base), tips acute, upper side color yellow-green, closest to but slightly more yellow than RHS 153D; under side color yellow-green, RHS 153D. Tips on both sides greyed-purple, RHS 183B. Lower bracts yellow-green, between RHS 146A and RHS 146B.

General shape/Arrangement.—Broad lanceolate, arranged alternately, tips bent downwards in an average angle of 40° to 85° (0°=horizontal).

Number.—Terminal Bracts: Average 9 Primary Bracts: Average 9 Scape Bracts: Average 9.

Texture.—Smooth, not glossy, not a very dull appearance.

Margin.—Entire.

Scape.—Length average 15 cm; width average 1.2 cm (measured approximately ⅓ from the base of the primary bracts).

Branch Spikes: Spikes (scapes) unbranched

Inflorescence:

Borne.—Inflorescences placed vertical, tops approximately 7 cm from top of bract, only very few inflorescences appear; average length of inflorescence 5 cm.

Individual flowers.—Average length 2.5 cm; average width 0.4 cm.

Calyx.—Average length 2.5 cm; white, RHS 155D.

Corolla.—Average length 1 cm; yellow, RHS 12A.

Time of blooming.—Summer.

Duration of inflorescence.—More than one month.

Reproductive Organs:

Ovary.—Ovate, average length 5 mm; average width 3 mm; light yellow-green in color, RHS 150D to green-white, RHS 157A. Ovary ribbed axially, due to three locules.

Style.—Average length 4 mm; light yellow-green in color, RHS 150D, stigma cleft in two parts, approximately 0.5 mm long.

Stamens.—Average 6, basifixed, shaped narrow sagittate, average length 6 mm; average width 1 mm; green-white in color, RHS 157A.

Roots: Thin, very well-branched, strong to moderately strong; color greyedorange, RHS 165A

Seed Characteristics: No fruits or seeds observed to date

I claim:

1. A new and distinct Guzmania plant named ‘Deplakar’ as described and illustrated herein.
